(19) United States
(12) Patent Application Publication
(10) Pub. No.: US 2017/0014487 A1
(43) Pub. Date: Jan. 19, 2017

(54) Method for treating ocular inflammation

(71) Applicant: THE TRUSTEES OF COLUMBIA UNIVERSITY IN THE CITY OF NEW YORK, New York, NY (US)

(72) Inventors: Martha WELCH, New York, NY (US); Bryan J. WINN, Mahwah, NJ (US)

(73) Assignee: The Trustees of Columbia University in the City of New York, New York, NY (US)

(21) Appl. No.: 15/123,801

(22) PCT Filed: Mar. 4, 2015
(86) PCT No.: PCT/US15/18830

§ 371 Date: Sep. 6, 2016

Related U.S. Application Data
(60) Provisional application No. 61/947,680, filed on Mar. 4, 2014.

(51) Int. Cl. A61K 38/22; A61K 45/06; A61K 9/00; A61K 38/11; A61K 39/00

(52) U.S. Cl. CPC A61K 38/2235; A61K 38/11; A61K 39/0008; A61K 9/0048; A61K 45/06; A61K 2039/54

(57) ABSTRACT

It has been discovered that the neuropeptides secretin, oxytocin and vasopressin can be administered as therapeutic agents alone or in various combinations to treat dry eye and dry mouth. Based on these discoveries and the results described, certain claims are directed to pharmaceutical compositions formulated for topical oral or ophthalmic use comprising therapeutically or prophylactically effective amounts of the neuropeptides secretin, oxytocin and secretin alone, or in various combinations.

### CROSS-REFERENCE TO RELATED APPLICATIONS

This application claims benefit of Provisional Appln. 61/947,680, filed on Mar. 4, 2014, the entire contents of which are hereby incorporated by reference as if fully set forth herein, under 35 U.S.C. §119(e).

### STATEMENT OF GOVERNMENTAL INTEREST

This invention was not made with government support.

### BACKGROUND

Over 20 million adults in the United States are afflicted with dry eye syndrome (DES), also known as keratoconjunctivitis sicca (KCS) or keratitis sicca (1). The eye depends on the flow of tears to provide constant moisture and lubrication to maintain vision and comfort. Tears are a combination of water, for moisture; oils, for lubrication; mucus, for even spreading; and antibodies and special proteins, for resistance to infection. These components are secreted by special glands located around the eye. When there is an imbalance in this tear system, a person may experience dry eyes. This irritating and painful condition has symptoms ranging from itching, burning, tearing, and blurred vision to devastating sequelae including corneal scarring and permanent vision loss. Symptoms include eye redness, a yellow or greenish discharge, ulceration of the cornea, pigmented cornea, and blood vessels on the cornea. Diagnosis is made by measuring tear production with a Schirmer tear test. Less than 15 millimeters of tears produced in a minute is abnormal. DES may be subdivided into 2 main types: DES associated with Sjogren syndrome (SS) and DES unassociated with SS (non-SS KCS). Its prognosis shows considerable variance, depending upon the severity of the condition. Most patients have mild-to-moderate cases, and can be treated symptomatically with wetting and lubricating eye drops.

Blepharitis, chronic eyelid inflammation, is one of the leading causes of evaporative dry eye, and it is seen in nearly one-half of ophthalmology visits (4). Recent studies have linked blepharitis to other chronic inflammatory/autoimmune conditions, including gastritis, peptic ulcer disease, asthma, and inflammatory bowel disease (5). While persons with autoimmune diseases have a high likelihood of having dry eyes, most persons with dry eyes do not have an autoimmune disease. Instances of Sjogren syndrome associated with DES are present much more commonly in women, with a ratio of 9:1. Milder forms of DES are also more common in women, partly because hormonal changes, such as those that occur in pregnancy, menstruation, and menopause, can decrease tear production. In areas of the world where malnutrition is common, vitamin A deficiency is a common cause of DES. Racial predilections do not exist for this disease.

To date, treatment of dry eye syndrome has remained elusive, and consists primarily of the supportive care of artificial tears, lid scrubs, and warm compresses. Corticosteroids and cyclosporine are partially effective in reducing the clinical signs and symptoms of DES (2, 3) but have significant ocular complications such as cataract formation and elevated intraocular pressure leading to glaucoma. Topical cyclosporin (topical cyclosporin A, tCSA) 0.05% ophthalmic emulsion is an immunosuppressant, marketed in the United States by Allergan under the trade name Restasis®. Restatis®, approved by the U.S. Food and Drug Administration in 2002, decreases surface inflammation presumably through inhibition of transcription factors required for cytokine production and T-lymphocyte maturation. Other treatments include temporary punctal occlusion which involves closing the ducts that drain tears out of the eye. This may be done temporarily with a dissolving plug that is inserted into the tear drain of the lower eyelid to determine whether permanent plugs can provide an adequate supply of tears. Non-dissolving punctal plugs and punctal occlusion by cautery (application of heat to tear exit duct) is often done if temporary plugging of the tear drains works well. These measures increase the tear level by blocking the “drainpipe” through which tears normally exit the eye and enter the nose. The plugs can be easily removed. More details on DES and blepharitis are presented below.

### SUMMARY OF THE INVENTION

It has been discovered that the neuropeptides secretin, oxytocin and vasopressin, can be administered as therapeutic agents alone or in various combinations to treat dry eye and dry mouth. Based on these discoveries and the results described here, certain claims are directed to pharmaceutical compositions formulated for topical oral or ophthalmic use comprising a therapeutically or prophylactically effective amount of secretin, or a combination of oxytocin and secretin, or a combination of oxytocin and vasopressin and secretin, or a combination of oxytocin and vasopressin, or a combination of secretin and vasopressin, or biologically active fragments, analogs or derivatives thereof, for the treatment of dry mouth or dry eye. In some embodiments the therapeutically or prophylactically effective amount of secretin, oxytocin, or vasopressin or biologically active fragment, analog or variant thereof in the pharmaceutical formulation/composition is in the range of from about 0.0001 to 0.005 mg/ml, 0.005 to 0.05 mg/ml, 0.05 to 0.5 mg/ml, 0.5 to 1 mg/ml, and 1 to 5 mg/ml. On other embodiments the pharmaceutical compositions further comprises a tear substitute or other lubricant, or at least one antiallergenic agent or a combination of at least one antiallergenic agent and a tear substitute or other lubricant. In some embodiments the pharmaceutical composition/formulation is a solution or an ointment.

Other embodiments are directed to various methods for treating dry eye or dry mouth. In an embodiment dry eye is treated by administering to the eye surface of the subject a therapeutically or prophylactically effective amount of secretin, or a combination of oxytocin and secretin, or a combination of oxytocin and vasopressin and secretin, or a combination of oxytocin and vasopressin, or a combination of secretin and vasopressin or biologically active fragments, analogs or derivatives thereof. In another embodiment dry mouth is treated by administering to the oral cavity of the subject a therapeutically or prophylactically effective amount of secretin, or oxytocin, or vasopressin, or a combination thereof or biologically active fragments, analogs or derivatives thereof, wherein the combinations include a combination of oxytocin and secretin, or a combination of oxytocin and vasopressin and secretin, or a combination of oxytocin and vasopressin, or a combination of secretin and vasopressin.

In certain embodiments the therapeutic agent(s) for treating dry eye or dry mouth is/are administered multiple times per day, either in a single pharmaceutical composition or in multiple compositions. The therapeutically effective amount of the neuropeptides secretin, oxytocin and vasopressin are administered in an amount from about 0.0001-0.005 mg/ml, 0.005 to 0.05 mg/ml; 0.05 to 0.5 mg/ml, 0.5 to 1 mg/ml, and 1 to 5 mg/ml.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1. Immunofluorescence staining for oxytocin receptors (OTR) in rat eyelid margin and conjunctiva.

FIG. 2. High power field of polymorphonuclear neutrophils (PMNs) in rat conjunctiva. Rat A received scopolamine but no treatment for dry eye compared to Rat B, which received scopolamine and combined secretin/oxytocin (S/OT) eye drops. PMNs are marked with yellow triangles.

FIG. 3. PMN density and confidence intervals for various dry eye treatment groups. “Scopolamine+Drug X drop” group has significantly fewer PMNs compared to the adjacent control group “Scopolamine+Saline drop.”

FIG. 4. A. Immunofluorescent detection of oxytocin receptors in the plasma membrane of SV40-HCEC corneal cell line. B. Western blot showing secretin receptor in SV40-HCEC cells.

FIG. 5. A. Western blot shows that TNF-α induces expression of ICAM-1 as a surrogate for neutrophil recruitment (inflammation) in SV40-HCEC cells β-actin serves as a loading control; B. addition of oxytocin and secretin to SV40-HCEC cells reduced expression of ICAM-1 about two-fold.

### DETAILED DESCRIPTION

It has been discovered that therapeutically or prophylactically effective amounts of the neuropeptide secretin administered topically to the eye by itself, or in various combinations with oxytocin and vasopressin, can be used to treat any form of dry eye including ocular inflammation such as DES and blepharitis. It has also been discovered that these same compounds and combinations can be administered topically to the oral cavity in therapeutically effective amounts to treat dry mouth, which can also be treated by topical administration of oxytocin or vasopressin alone. Certain embodiments are directed to methods of treating dry eye and dry mouth syndrome, and to pharmaceutical compositions comprising these therapeutic agents

### 1. DEFINITIONS

Unless defined otherwise, technical and scientific terms used herein have the same meaning as commonly understood by one of ordinary skill in the art to which this invention belongs. The practice of the present invention will employ, unless indicated specifically to the contrary, conventional methods of molecular biology and recombinant DNA techniques within the skill of the art, many of which are described below for the purpose of illustration. Such techniques are fully explained in the literature. See, e.g., Singleton et al., Dictionary of Microbiology and Molecular Biology 3rd.sup.ed., J. Wiley & Sons (2001); March, Advanced Organic Chemistry Reactions, Mechanisms and Structure 5th.sup.ed., J. Wiley & Sons (2001); Sambrook & Russell, eds., Molecular Cloning: A Laboratory Manual 3rd ed., Cold Spring Harbor Laboratory Press (2001); Glover, ed., DNA Cloning: A Practical Approach, vol. I & II (2002); Gait, ed., Oligonucleotide Synthesis: A practical approach, Oxford University Press (1984); Herdewijn, ed., Oligonucleotide Synthesis: Methods and Applications, Humana Press (2005); Hames & Higgins, eds., Nucleic Acid Hybridisation: A Practical Approach, IRL Press (1985); Buzdin & Lukyanov, eds., Nucleic Acid Hybridization: Modern Applications, Springer (2007); Hames & Higgins, eds., Transcription and Translation: A Practical Approach, IRL Press (1984); Freshney, ed., Animal Cell Culture, Oxford UP (1986); Freshney, Culture of Animal Cells: A Manual of Basic Technique and Specialized Applications, 6th ed., John Wiley & Sons (2010); Perbal, A Practical Guide to Molecular Cloning, 3rd ed., Wiley-Liss (2014); Farrell, RNA Methodologies: A Laboratory Guide for Isolation and Characterization, 3rd ed., Elsevier/Focal Press (2005); Lilley & Dahlberg, eds., Methods in Enzymology: DNA Structures, Part A: Synthesis and Physical Analysis of DNA, Academic Press (1992); Harlow & Lane, Using Antibodies: A Laboratory Manual: Portable Protocol no. 1, Cold Spring Harbor Laboratory Press (1999); Harlow & Lane, eds., Antibodies: A Laboratory Manual, Cold Spring Harbor Laboratory Press (1988); Seethala & Fernandes, eds., Handbook of Drug Screening, Marcel Dekker (2001); and Roskams & Rodgers, eds., Lab Ref: A Handbook of Recipes, Reagents, and Other Reference Tools for Use at the Bench, Cold Spring Harbor Laboratory (2002), which provide one skilled in the art with a general guide to many of the terms used in the present application.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

“Blepharitis” refers to an inflammation of the eyelids causing red, irritated, itchy eyelids and the formation of dandruff-like scales on eyelashes. It is a common eye disorder caused by either bacteria or a skin condition such as dandruff of the scalp or acne rosacea. It affects people of all ages. Although uncomfortable, blepharitis is not contagious and generally does not cause any permanent damage to eyesight. Blepharitis as used herein is a form of ocular inflammation.

“Arginine vasopressin (AVP),” also referred to herein as “vasopressin”, argipressin or antidiuretic hormone (ADH), is a neurohypophysial hormone found in most mammals. Its two primary functions are to retain water in the body and to constrict blood vessels. It also increases peripheral vascular resistance, which in turn increases arterial blood pressure. It plays a key role in homeostasis. The amino acid sequence of AVP is CYFQNCPRG-NH₂.

As used herein, “conjunctiva” means the tissue that lines the inside of the eyelids and covers the sclera. It is composed of non-keratinized, stratified columnar epithelium with goblet cells. The conjunctiva helps lubricate the eye by producing mucus and tears. It also contributes to immune surveillance and helps to prevent the entrance of microbes into the eye. Inflammation of the cornea is referred to as “conjunctivitis.”

As used herein, “secretin” refers to the neuropeptide secretin and analogues and derivatives thereof, including, for example, natural or synthetic functional variants which have secretin biological activity, as well as fragments of secretin having secretin biological activity. As further used herein, the term “secretin biological activity” refers to activity that causes pancreatic secretion of bicarbonate and enzymes, and inhibits gastrin release from the stomach. Secretin intermediates and derivatives for use in the invention are described inter alia in Guiducci, et al. U.S. Pat. No. 3,987,014, including [6-TYR]. Secretin was found to be biologically active by the tests described by Jorpes et al., Acta. Chem. Scand. 15, 1970 (1961); Biochem. Biophys. Res. Commun. 9, 275 (1962); Biochem 4, 2358 (1965); and Fourth Int. Symp. Chem. Nat. Prod., Stockholm (1966). Secretin further includes a biologically active fragments, analogs and derivatives/variants thereof. Homo sapiens secretin (SCT), mRNA is listed at NCBI Reference Sequence: NM_021920.2, and secretin preproprotein [Homo sapiens] is listed at NCBI Reference Sequence: NP_068739.1,

As used herein the term “derivatives” includes variants of the neuropeptides that have the biological activity of reducing one or more symptoms of dry eye or dry mouth.

As used herein, “oxytocin” refers to the nonapeptide oxytocin (Oxt) (OT), a mammalian neurohypophysial hormone that is a posterior pituitary hormone synthesized as an inactive precursor in the hypothalamus along with its carrier protein neurophysin I. Oxytocin has the systematic name cysteine-tyrosineisoleucine-glutamine-asparagine-cysteine-proline-leucine-glycine-amide or CYIQNCPLG-NH 2), and a molecular mass of 1007 daltons. One international unit (IU) of oxytocin is the equivalent of about 2 micrograms of pure peptide. While the structure of oxytocin is highly conserved in placental mammals, a novel structure of oxytocin was recently reported in marmosets, tamarins, and other new world primates. The term “Oxytocin” as used here includes the neuropeptide oxytocin and analogues and derivatives thereof, including, for example, natural or synthetic functional derivatives/variants which have oxytocin biological activity, as well as fragments of oxytocin having oxytocin biological activity. A biologically active form of oxytocin is also known as the octapeptide “oxytocin disulfide” (oxidized form). As further used herein, the term “oxytocin biological activity” refers to activity that reduces any symptom of dry eye syndrome or dry mouth syndrome, or causes an anti-inflammatory response. Oxytocin's chemical name is 1-({(4R,7S,10S,13S,16S,19R)-19-amino-7-(2-amino-2-oxoethyl)-10-(3-amino-3-oxopropyl)-16-(4-hydroxybenzoyl)-13-[(1S)-1-methylpropyl]-6,9,12,15,18-pentaoxo-1,2-dithia-5,8,11,14,17-pentaazacycloicosan-4-yl}carbonyl)-L-prolyl-L-leucylglycinamide.

The term “neuropeptide” as used herein collectively refers to oxytocin, secretin and vasopressin.

The term “aqueous” typically denotes an aqueous composition wherein the carrier is to an extent of >50%, more preferably >75% and in particular >90% by weight water.

As used herein, the term “antiallergenic agent” refers to a molecule or composition that treats ocular allergy or reduces a symptom of ocular allergy. Examples of antiallergenic agents include, but are not limited to, “antihistamines” or drugs that block histamine from binding to the histamine receptors, “mast cell stabilizers” or drugs that block the release of histamine and other substances from the mast cell, “drugs with multiple modes of action” or drugs that are antiallergenic agents having multiple modes of action (e.g. drugs that are antihistamines and mast cell stabilizers, drugs with antihistamine, mast cell stabilizing and anti-inflammatory activity, etc.), and “nonsteroidal anti-inflammatory drugs” or NSAIDs.

The term “dry eye” (also referred to as xerophthalmia) as used herein includes any condition resulting in irritation or a sensation of dryness of the eye, including any type of ocular surface inflammation and it includes any disease or disorder or condition which results in an adverse effect on the quality of the tear film that lubricates the eyes, regardless of etiology. The disease or disorder may be of the eye itself, or of another part of the body, so long as it results in an adverse effect on the quality of the tear film that lubricates the eyes. Examples of dry eye are set forth in more detail below.

The term “dry mouth” as used herein (also known as xerostomia or dry mouth syndrome) is the medical term for the subjective symptom of dryness in the mouth, which may be associated with a change in the composition of saliva or reduced salivary flow (hyposalivation) or have no identifiable cause, regardless of the etiology. A result of reduced or no saliva, dry mouth can lead to problems because saliva helps prevent tooth decay by limiting bacterial growth and washing away food particles. Saliva also enhances the ability to taste and makes it easier to swallow. In addition, enzymes in saliva aid in digestion. Dry mouth is very common and is often seen as side effect of many types of medication. It is more common in older people (mostly because members of this group tend to take several medications) and in people who breathe through their mouths (mouth breathing). Dehydration, radiotherapy involving the salivary glands, and several diseases including inflammatory diseases and autoimmune diseases can cause hyposalivation or a change in saliva consistency leading to dry mouth.

The phrase “effective amount” is an art-recognized term, and refers to an amount of an agent that, when incorporated into a pharmaceutical compositions of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a symptom of dry eye, or prevent or treat dry eye or dry mouth syndromes. The effective amount may vary depending on such factors as the disease or condition being treated, the particular composition being administered, or the severity of the disease or condition. One of skill in the art may empirically determine the effective amount of a particular agent without necessitating undue experimentation.

The term “neuropeptide” as used herein means a peptide with a direct synaptic effect (i.e., a peptide that is a neurotransmitter) and/or an indirect effect on synaptic transmission. Neuropeptides may be released from neurons or from non-neuronal cells, and may also act as hormones.

The term “ocular allergy” as used herein refers to any allergic disease of the eye. Examples of such ocular allergies include but are not limited to seasonal/perennial allergic conjunctivitis, vernal keratoconjunctivitis, giant papillary conjunctivitis, perennial allergic conjunctivitis and atopic keratoconjunctivitis. The signs and symptoms of ocular allergies include chemosis, eye itching, redness and swelling.

The phrase “pharmaceutically acceptable” is art-recognized and refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “tear substitute” refers to molecules or compositions which lubricate, “wet,” approximate the consistency of endogenous tears, aid in natural tear build-up, or otherwise provide temporary relief of dry eye symptoms and conditions upon ocular administration.

The term “treating” is an art-recognized term which refers to curing as well as ameliorating at least one symptom of any condition or disease.

“Administering” or “administration of” a drug or therapeutic pharmaceutical composition to a subject as used herein means any method of administering a topical formulation to the eye or oral cavity to treat dry eye or dry mouth, respectively.

Administration of an agent “in combination with” includes parallel administration of two agents to the patient over a period of time, co-administration (in which the agents are administered at approximately the same time, e.g., within about a few minutes to a few hours of one another), and co-formulation (in which the agents are combined or compounded into a single dosage form suitable for oral, subcutaneous or parenteral administration).

A “subject” is a mammal, typically a human, but optionally a mammalian animal of veterinary importance, including but not limited to horses, cattle, sheep, dogs, and cats. In some embodiments a “subject” refers to either one who has been previously diagnosed with or identified as suffering from dry eye or dry mouth or both.

“Vasoconstrictors” are drugs that cause vasoconstriction; they are also called vasopressors, or simply “pressors.”

“Ocular inflammation” is any inflammation of the eye, including uveitis. Uveitis is, broadly, inflammation of the uvea. The uvea consists of the middle, pigmented vascular structures of the eye and includes the iris, ciliary body, and choroid.

“Therapeutic Agents” and “Active Agents” refer to the neuropeptides secretin, vasopressin and oxytocin, and biologically active fragments or analogs or derivatives/variants thereof.

“Biologic Activity” refers to the ability of the active agent, fragment or variant to reduce or ameliorate one or more symptoms of dry eye or dry mouth in a subject.

“A biologically active fragment or variant” of an active agent (oxytocin, secretin and vasopressin) refers to activity that reduces one or more symptoms of dry eye syndrome or dry mouth syndrome, or causes an anti-inflammatory response.

### 2. OVERVIEW

Inflammatory cytokines are present in high concentrations in the tears of patients with blepharitis (8). Rat models of dry eye syndrome also show increased expression of these cytokines in the surface of the eye (9). Further, it has been shown in mice that a mutation disrupting this cytokine signaling pathway resulted in milder dry eye disease compared to wild-type mice when both groups were exposed to a desiccating agent (10).

Elevated levels of inflammatory cytokines such as TNF-α, IL-6, interferon-Υ(INF-Υ), IL-17A and IL-2 have been shown to play a role in inflammatory bowel disease (IBD) (6). It has been reported that the neuropeptides oxytocin and secretin administered together significantly reduced the pro-inflammatory cytokines TNF-α and INF-γ in an experimental rat model of IBD (7).

Others have reported the use of various neuropeptides including oxytocin for treating dry eye syndrome (Ousler, George W. III et al., U.S. Patent Apn. Publication No. 20060270592), but none have described the use of secretin, either alone or in combination with oxytocin for treating ocular inflammation such as dry eye syndrome. Experiments were conducted to determine whether the combination of oxytocin and secretin would affect ocular inflammation.

### 3. RESULTS

### Oxytocin Receptors are Present in the Conjunctiva and Corneal Epithelium of the Rat Eye

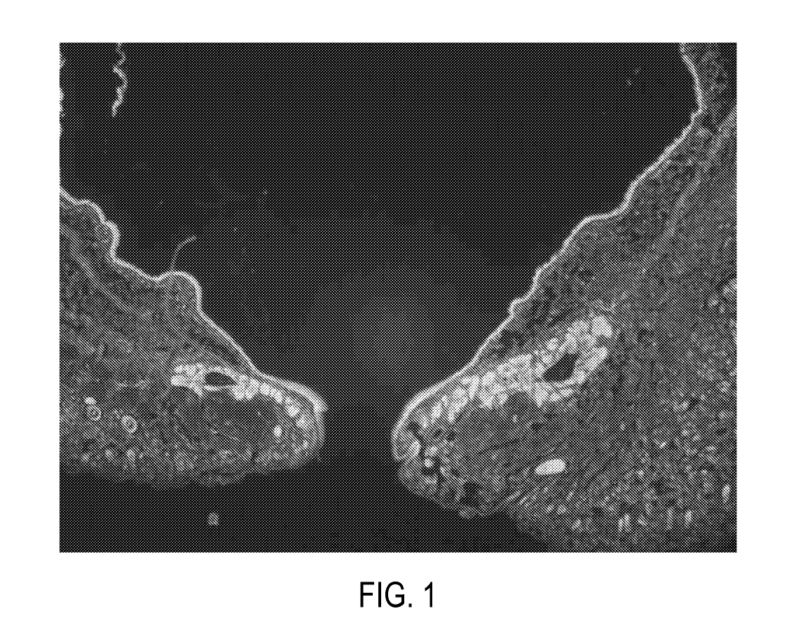

Immunofluorescence staining for oxytocin receptors (OTR) showed that OTR are present in the conjunctiva and corneal epithelium of the rat eye (see FIG. 1).

Combination Treatment of Dry Eye with Secretin/Oxytocin (S/OT) Reduced Inflammation 50%

Previous studies have shown that rats treated with subcutaneous injections or continuous infusions of scopolamine are a suitable rat model for dry eye syndrome (11). Therefore this model was used in the experiments described herein. A small trial involving 3-5 rats per arm of a 6-armed pilot study was conducted to investigate the role of combination treatment with secretin/oxytocin (S/OT) administered by drops in a single formulation in an amount of 50 μg of oxytocin and 50 μg secretin per 1 mL (0.005% oxytocin and 0.005% secretin combination) to treat inflammation in the mouse model for dry eye syndrome. Elevated numbers of white blood cells (polymorphonucleocytes or PMNs) in the conjunctiva was used as an indication of the extent of inflammation (e.g. controls 0-5 PMNs versus dry eye animals >50 PMNs).
The experimental conditions consisted of:
True controls untreated rats
Dry eye controls scopolamine-induced dry eye rats, with no other treatment
Dry eye/saline rats scopolamine-induced dry eye with saline drop treatment
Dry eye S/OT rats scopolamine-induced dry eye with combined secretin and oxytocin (S/OT) treatment

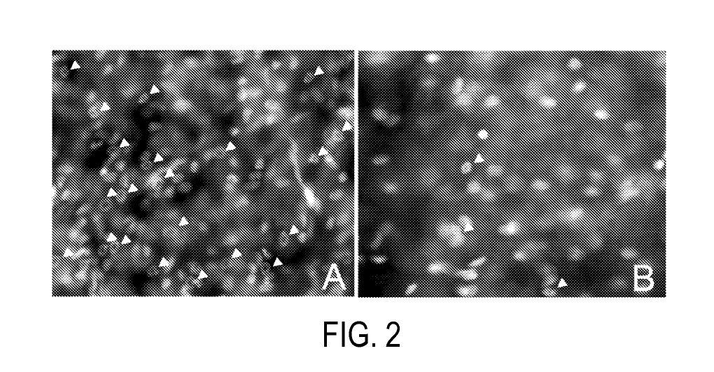

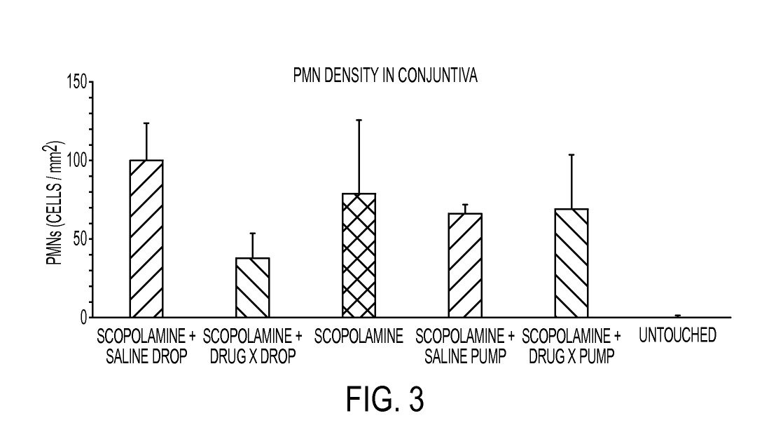

In the study, true control rats (untouched) which were not subjected to the dry eye-inducing effects of scopolamine showed virtually no signs of ocular surface inflammation. (See FIG. 2 and Table 1.) In contrast, dry eye controls (rats treated with scopolamine only, with no drops or other infusions) had significant ocular surface inflammation as was demonstrated by elevated numbers of PMNs (polymorphonucleocytes, inflammatory white blood cells) in the conjunctiva. Untreated control animals that were not subjected to scopolamine-induced dry eye syndrome (“true controls”) typically had 0-5 PMNs/mm2 in the conjunctiva, whereas animals subjected to scopolamine but without treatment (“dry eye controls”) always had greater than 50 PMNs/mm2. (See FIG. 2A.) Dry eye/saline rats treated with scopolamine and topical saline drops had similarly high levels of conjunctival PMNs. However, Dry eye S/OT had over 50% fewer conjunctival PMNs compared to both dry eye/saline rats and dry eye controls, indicating a significant reduction in inflammation with the combination neuropeptide treatment. (See FIG. 2B and Table 1.) As shown in FIG. 3, the group labeled “Scopolamine+Saline drop” has significantly more inflammation indicated by a high PMN count compared to the group labeled “Scopolamine+Drug X drop” (p=0.002) receiving topical S/OT. FIG. 3 further shows that systemic administration of S/OT was not effective in reducing inflammation. Mean density of conjunctival PMNs and standard deviations are shown in Table 1.

TABLE 1
Mean density of conjunctival inflammatory cells (PMNs)
and standard deviations. Drug X represents S/OT.

PMNs/mm2

| | | Avg | SD |
|---|---|---|---|
| Conj | Scopolamine + Saline drop | 100 | 24 |
| Conj | Scopolamine + Drug X drop | 38 | 16 |
| Conj | Scopolamine | 79 | 48 |
| Conj | Scopolamine + Saline pump | 67 | 5 |
| Conj | Scopolamine + Drug X pump | 70 | 35 |
| Conj | Untouched | 1 | 1 |

### SV40-HCEC Plasma Membrane Expresses Oxytocin Receptors

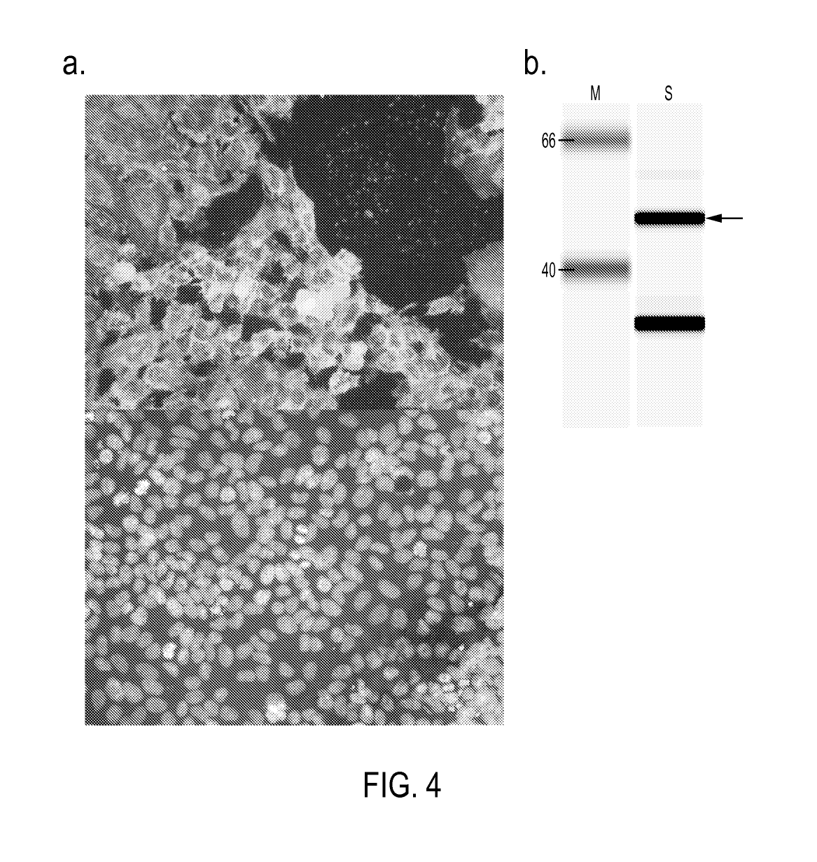

Experiments were conducted to set up an in vitro model for dry eye syndrome, using cell cultures of immortalized human cells from the cornea or conjunctiva using the SV40-HCEC corneal cell line of SV40 virus-immortalized corneal cells (12). SV40-HCEC cells were shown using immunofluorescence of anti-oxytocin receptor antibodies to contain oxytocin receptors in the plasma membrane (FIG. 4A, oxytocin receptor fluorescence is in red [top panel]; blue represents nuclear staining with DAPI stain [bottom panel]), consistent with the results showing its presence in rodent conjunctiva and cornea. Moreover, the presence of secretin receptors in SV40HCEC cells was shown using Western blot (FIG. 4B, the presence of secretin receptor migrating at the expected size [arrow]). M: Marker, S: Sample/secretin receptor. The lower band in the sample lane represents degradation product.

Combination Treatment of Dry Eye with Secretin/Oxytocin (S/OT) Reduced ICAM-1 Expression

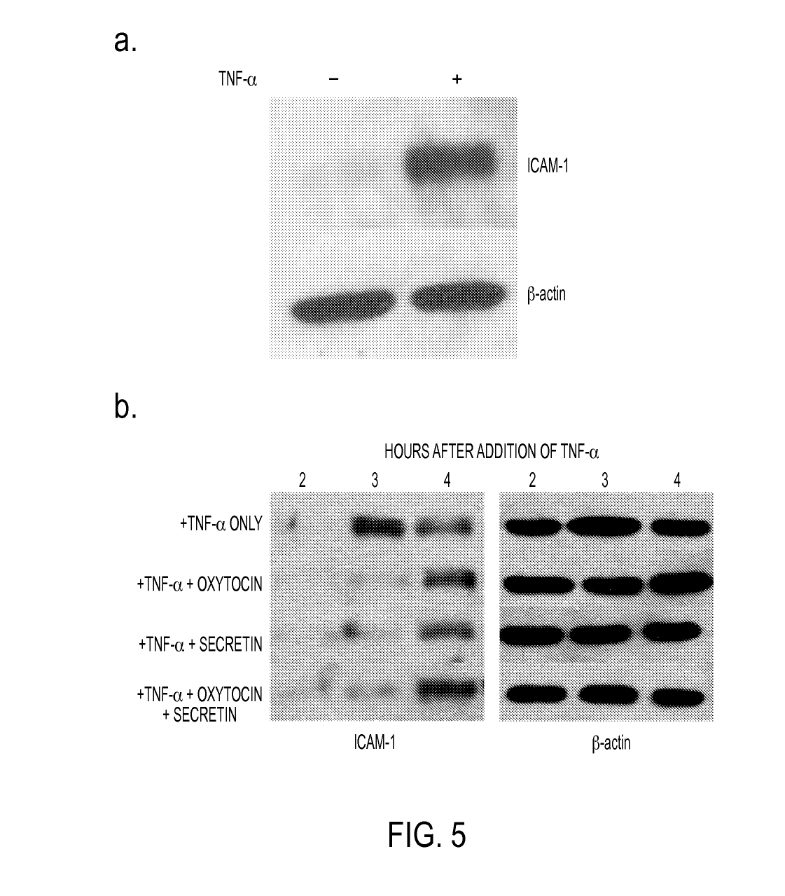

A Western blot-based assay was developed to assess for the role of oxytocin and secretin in dry eye syndrome in vitro. The expression of the integrin ICAM-1 in SV40-HCEC cells was induced by adding the pro-inflammatory cytokine TNF-α [13]. In vivo, ICAM-1 acts to recruit neutrophils to sites of inflammation, and thus acts as a surrogate for inflammation in vitro. Experiments show that addition of TNF-α induced expression of ICAM-1 in SV40-HCEC cells (FIG. 5A); and the addition of 100 μM oxytocin, 1-μM secretin or a combination of oxytocin and secretin all reduced the rate of accumulation of ICAM-1 about two-fold, showing that they reduce inflammation.

### Embodiments

Based on the results described herein, certain embodiments are directed to novel methods for treating any form of dry eye (including ocular inflammation including DES and blepharitis) by administering a therapeutically effective amount of secretin; or combinations of oxytocin and secretin; oxytocin, vasopressin and secretin; oxytocin and vasopressin, and secretin and vasopressin. Preferred administration is topically to the eye.

Other embodiments are directed to novel methods for treating any form of dry mouth by administering a therapeutically effective amount of secretin; vasopressin or oxytocin individually; or combinations of oxytocin and secretin; oxytocin, vasopressin and secretin; oxytocin and vasopressin, and secretin and vasopressin. Preferred administration is topically to the oral cavity.

The terms “pharmaceutical compositions” and “formulations” are used interchangeably herein. Other embodiments are directed to pharmaceutical compositions for topical ocular administration or topical oral administration comprising therapeutically or prophylactically effective amounts of secretin; oxytocin and secretin; oxytocin, vasopressin and secretin; oxytocin and vasopressin; and secretin and vasopressin. Pharmaceutical formulations are intended to treat any form of dry eye (including ocular inflammation including DES and blepharitis) or dry mouth or reduce one or more symptoms thereof. Other embodiments are directed to methods of treating or preventing dry eye or dry mouth by administering a therapeutically effective or prophylactically effective amount of a pharmaceutical composition described above. Such formulations can be liquid drops, eye or mouth washes, or sprays suitable for ophthalmic or oral administration.

### 4. DRY EYE AND DRY MOUTH SYNDROME

Diseases that affect the ability to make tears, such as autoimmune conditions (including Sjogren's syndrome and rheumatoid arthritis) and other collagen vascular diseases, can cause dry eye. Dry eye includes ocular inflammatory conditions that include: (i) Eyelid inflammations: blepharitis, chronic eyelid edema, meibomitis, ocular rosacea, thyroid eye disease; (ii) Conjunctival inflammations: chronic papillary conjunctivitis, chronic follicular conjunctivitis, nonspecific chronic conjunctivitis, giant papillary conjunctivitis, ocular cicatricial pemphigoid, cicatrizing conjunctivitis, allergic conjunctivitis, phlyctenular corneoconjunctivitis; (iii) Corneal inflammations: dry eye syndrome, ocular rosacea, superficial punctate keratitis, infectious keratitis, peripheral ulcerative keratitis, Thygeson's superficial punctate keratitis, corneal graft rejection, disciform keratitis, stromal keratitis; (iv) Uveitis: Anterior uveitis, HLA-B27 uveitis, Behcet's disease, atopic eye disease, juvenile rheumatoid arthritis, pars planitis, sarcoidosis, Vogt-Koyanagi-Harada syndrome, sympathetic ophthalmia, Fuch's heterochromic iridocyclitis, glaucomatocyclitis crisis (Posner-Schlossman syndrome); and (v) Retinal inflammations (not already mentioned): chronic macular edema, central serous choreoretinopathy, white dot syndrome, acute retinal necrosis. Causes include idiopathic, diabetes, congenital alacrima, xerophthalmia, lacrimal gland ablation, and sensory denervation as well as other conditions, factors and phenomena such as prolonged contact lens wear, advanced age, circulating hormones, allergies, ocular surgeries including PRK or LASIK, many medications, environmental conditions, visual tasking such as computer use, ocular fatigue, mechanical influences such as corneal sensitivity, partial lid closure, surface irregularities (e.g. pterygium), and lid irregularities (e.g. ptosis, entropion/ectropion, pinguecula). In rare cases, it may be a symptom of collagen vascular diseases, including rheumatoid arthritis, Wegener's granulomatosis, cystic fibrosis, and systemic lupus erythematosus.

The following are the most common complaints associated with dry eye syndrome (DES): Foreign-body sensation and ocular dryness and grittiness; hyperemia mucoid discharge; ocular irritation; excessive tearing (secondary to reflex secretion); photophobia; and fluctuating or blurry vision.

Drugs such as isotretinoin, sedatives, diuretics, tricyclic antidepressants, antihypertensives, oral contraceptives, antihistamines, nasal decongestants, beta-blockers, phenothiazines, atropine, and pain relieving opiates such as morphine can cause or worsen this condition. Infiltration of the lacrimal glands by sarcoidosis or tumors, or postradiation fibrosis of the lacrimal glands, can also cause this condition.

Tests that may be used for diagnosis of dry eye syndrome include the following: impression cytology (e.g., to monitor the progression of ocular surface changes), measurement of tear breakup time (TBUT), the Schirmer test, and quantification of tear components (e.g., through analysis of tear proteins or tear-film osmolarity). Additional tests that may be used in workup include the tear stability analysis system (TSAS), the tear function index (TFI; Liverpool modification), and the tear ferning test (TFT). Criteria for a diagnosis of dry eye syndrome associated with Sjogren syndrome (SS) include an abnormally low Schirmer test result, objective evidence of low salivary flow, biopsy-proven lymphocytic infiltration of the labial salivary glands, and dysfunction of the immune system, as manifested by the presence of serum autoantibodies (e.g., antinuclear antibody [ANA], rheumatoid factor [RF], and anti-Ro [SS-A] and anti-La [SS-B] antibodies).

Lubricating supplements are the medications most commonly used to treat DES. Agents that have been used to treat DES include the following: Rebamipide, a mucin secretagogue; Artificial tear substitutes; gels and ointments; anti-inflammatory agents; topical cyclosporine, topical corticosteroids; or topical or systemic omega-3 fatty acids (omega-3 fatty acids inhibit the synthesis of lipid mediators and block the production of interleukin [IL]-1 and tumor necrosis factor alpha [TNF-α]). Topical or systemic tetracyclines have also been used. Other treatments include secretagogues such as Diquafosol (approved in Japan but not in the United States), autologous or umbilical cord serum, and systemic immunosuppressants.

Surgical intervention includes the use of punctal plugs that are often employed in the treatment of DES. Other surgical options include sealing of the perforation or descemetocele with corneal cyanoacrylate tissue adhesive; corneal or corneoscleral patching for an impending or frank perforation; lateral tarsorrhaphy/temporary tarsorrhaphy (50%) for patients with DES secondary to exposure keratitis after facial nerve paralysis and after trigeminal nerve lesions that give rise to DES secondary to loss of corneal sensation; conjunctival flap; surgical occlusion of the lacrimal drainage system; mucous membrane grafting; salivary gland duct transposition; and amniotic membrane transplantation.

Diseases associated with dry eye are also often associated with dry mouth, also called hyposalivation, as the tear ducts in the eye are connected to the mouth and both comprise mucosal cells. Causes of dry mouth include cancer therapy, as chemotherapy drugs can change the nature of saliva and the amount produced. This effect may be temporary, with normal salivary flow returning after treatment has been completed. Radiation treatments to the head and neck can damage salivary glands, causing a marked decrease in saliva production. This can be temporary or permanent, depending on the radiation dose and area treated. An injury or surgery that causes nerve damage to the head and neck area can result in dry mouth. Dry mouth can be a consequence of certain health conditions, including the autoimmune disease Sjogren's syndrome or HIV/AIDS. Stroke and Alzheimer's disease may cause a perception of dry mouth, even though the salivary glands are functioning normally. Snoring and breathing with the mouth open also can contribute to dry mouth. Smoking or chewing tobacco can increase dry mouth symptoms. Methamphetamine use can cause severe dry mouth and damage to teeth, a condition also known as “meth mouth.” More than 400 medicines can cause the salivary glands to make less saliva. Drugs that can cause dryness include antihistamines, decongestants, diuretics, some anti-diarrhea drugs, some anti-psychotic drugs, tranquilizers, some antihypertensives (e.g. terazosin, prazosin, clonidine, atenolol, propranolol), antidepressants (tricyclic antidepressants, selective serotonin reuptake inhibitors, lithium), and anti-reflux drugs (proton pump inhibitors, e.g. omeprazole), opioids, cytotoxic drugs, retinoids, bupropion, protease inhibitors, didanosine, diuretics, ephedrine, benzodiazepines and interleukin-2.

### 5. OXYTOCIN AND SECRETIN

A biologically active form of oxytocin, commonly measured by RIA and/or HPLC techniques, is also known as the octapeptide “oxytocin disulfide” (oxidized form). Oxytocin's actions are mediated by specific, high-affinity oxytocin receptors which belong to the rhodopsin-type (class I) group of G-protein-coupled receptors. Oxytocin also exists as a reduced dithiol nonapeptide called oxytoceine, which form may have use in the pharmaceutical embodiments of the invention. It has been theorized that open chain oxytoceine (the reduced form of oxytocin) may also act as a free radical scavenger (by donating an electron to a free radical); oxytoceine may then be oxidized back to oxytocin via the redox potential of dehydroascorbate ascorbate. For the purpose of embodiments of the invention, a biologically active fragment or analog or derivative/variant of oxytocin, (as well as secretin or vasopressin) is one which treats dry eye or dry mouth by reducing one or more symptoms of dry eye or dry mouth.

The structure of oxytocin is very similar to that of vasopressin; these are the only known hormones released by the human posterior pituitary gland to act at a distance. Oxytocin (OT) and vasopressin (AVP, arginine-vasopressin, antidiuretic hormone) are closely related, highly conserved, multifunctional neuropeptides that are mainly synthesized in the magnocellular and parvocellular neurons of the hypothalamus. OT and AVP are structurally very similar nonapeptides. In humans, OT acts via one oxytocin receptor (OTR) and AVP via three vasopressin receptors (AVPRs—vasopressor V_{1aR}, pituitary V_{1bR}, renal V_{2R}), all of which are members of the G-protein-coupled receptor family. OTRs and secretin receptors have now been discovered to be expressed on the corneal surface. The similarity of OT and AVP together with the high sequence homology of the extracellular binding domain of OTR and AVPRs results in significant cross talk, therefore in certain embodiments of the invention AVP is administered together with secretin and/or oxytocin to treat dry eye or dry mouth syndrome. The amino acid sequence of oxytocin is CYIQNCPLG-NH₂, and the amino acid sequence of AVP is CYFQNCPRG-NH₂

Oxytocin is commercially available from Fujisawa Healthcare, Inc., Three Parkway North, Deerfield, Ill. 60015-2548. Synthetic oxytocin is sold as proprietary medication under the trade names Pitocin and Syntocinon, and as generic oxytocin. It has a half-life of typically about three minutes in the blood, and given intravenously does not enter the brain in significant quantities. Oxytocin is relatively safe when used at recommended doses, and side effects are uncommon. Oxytocin, secretin and vasopressin, for use in the present invention, include natural, synthetic and recombinant forms.

Secretin is an endocrine hormone belonging to the glucagon family; its major site of production is the endocrine S cells located in the proximal small intestinal mucosa. The release of active secretin is stimulated by either fatty acids or an acidic pH in the duodenum. This hormone stimulates the secretion of bicarbonate-rich pancreatic fluids and has also been shown to regulate the growth and development of the stomach, small intestine, and pancreas. Secretin deficiency has been implicated in autistic syndrome, suggesting that the hormone could have a neuroendocrine function in addition to its role in digestion. Secretinergic cells have been identified in the forebrain.

Methods of preparing secretin and its analogues and derivatives are well known in the art. Secretin, for example, is commercially available as SecreFlo™ from RepliGen Corporation, 41 Seyon Street Building #1, Suite 100, Waltham, Mass. 02453 Moreover, both secretin and oxytocin may be obtained in accordance with known biochemical procedures that are readily understood by those of skill in the art.

The present invention also includes the use of peptide analogues of oxytocin, vasopressin and secretin. In an embodiment, certain peptide analogues are seleno-analogues of oxytocin in which the C-terminal amide has been replaced by a carboxylic acid. These peptides may have increased selectivity for the oxytocin receptor as compared to vasopressin receptors. Methods of synthesis of selenocysteine, tellurocysteine and oxytocin analogues are described in U.S. Patent Application Publication No. 20130130985, Alewood, Paul, et al.

Methods of preparing secretin and its analogues and variants or derivatives are well known in the art. Secretin, for example, is commercially available as SecreFlo™ from RepliGen Corporation, 41 Seyon Street Building #1, Suite 100, Waltham, Mass. 02453 Moreover, both secretin and oxytocin may be obtained in accordance with known biochemical procedures that are readily understood by those of skill in the art.

### 6. PHARMACEUTICAL COMPOSITIONS

In a method of the present invention, secretin is administered to a subject in need of treatment for dry eye or dry mouth either alone or in various combinations described above with oxytocin or vasopressin. In certain embodiments oxytocin or vasopressin are administered alone to treat dry eye or dry mouth or in various combinations described above with secretin. Administration of a therapeutic agent such as secretin “in combination with” another compound such as oxytocin and/or vasopressin refers to co-administration of at least two therapeutic agents that may occur concurrently, sequentially, or alternately. Concurrent co-administration refers to administration of both secretin and oxytocin at essentially the same time for example in a single, combined formulation, containing therapeutically effective amounts of both secretin and oxytocin in physical association with one another, preferably a topical formulation for either ophthalmic or oral administration. For concurrent co-administration, the courses of treatment with secretin and oxytocin may be run simultaneously. Treatment of dry eye/dry mouth may often require multiple topical applications of the pharmaceutical compositions of the invention per day, according to symptoms. For sequential co-administration, one of the therapeutic agents is separately administered, followed by the other.

Each therapeutic agent/neuropeptide in embodiments of the ophthalmic and oral pharmaceutical formulations is present at a concentration of from about 0.0001 mg/ml (i.e. 0.1 μg/ml) to about 5 mg/ml, including ranges of from 0.0001-0.005, 0.005 to 0.05; 0.05 to 0.5, 0.5 to 1, and 1 to 5 mg/ml. The lower ranges are useful for long term prophylactic administration or for testing for an adverse reaction, the intermediate ranges are account for patient variability (age, general health, etc.) and variations in the severity of the disorder (dry eye or dry mouth or combination thereof), and the highest concentrations are useful for the most severe conditions in subjects that tolerate the higher amounts without an adverse reaction.

The dose of the pharmaceutical formulation, administered therapeutically or prophylactically, depends on the concentration of the therapeutic agent in the formulation and the amount of the formulation applied. In an embodiment, two or three drops are administered to each eye. There are about twenty drops per ml; therefore, for example, two drops of a neuropeptide formulation of 60 ugm (micrograms) per ml oxytocin would be 6 ugm per two drops. Similarly, two drops of oxytocin at 600 ugm per ml would be 60 ugm per two drops, and so on. Actual doses range from 0.1 ugm per ml neuropeptide up to about 5 mg per ml and various ranges within. Similar calculations are used to determine the dose of pharmaceutical formulations of the invention applied to the mouth. The ophthalmic and oral pharmaceutical formulations of the invention can be administered either therapeutically or prophylactically including to very early stages of dry eye or dry mouth to minimize or prevent one or more symptoms of the disease. Low dose prophylactic treatment is particularly useful in subjects having a chronic predisposition to these disorders, for example, due to an autoimmune disorder. The prophylactic amount of the neuropeptide combinations may be less than the therapeutic amount, but it will be in the same concentration range of from about 0.0001 mg/ml to 5 mg/ml for each neuropeptide in the pharmaceutical formulations.

The therapeutic agents of the present invention (i.e., secretin, vasopressin and oxytocin) may be administered to a human or animal subject by known procedures, preferably topically to the eye or oral cavity. The therapeutic agents can be administered separately or in a single formulation. For example, an amount of the secretin may be packaged in a vial or unit dose, and an amount of the oxytocin may be packaged in a separate vial or unit dose. A combination of the secretin and the oxytocin then may be produced by mixing the contents of the separate vials or unit doses in vitro.

The dosage or therapeutically effective amount of the therapeutic neuropeptides in a formulation will depend on the concentration of the active therapeutic agent in the formulation, the amount of the formulation administered (i.e. how may eye drops are used) and on its rate of absorption, inactivation, and excretion. It is to be noted that dosage values may also vary with the severity of the condition to be alleviated. It is to be further understood that for any particular subject, specific dosage regimens should be adjusted over time according to the individual's need and the professional judgment of the person administering or supervising the administration of the compositions. Typically, dosing will be determined using techniques known to one skilled in the art.

The dosage/amount of therapeutic agent administered may vary depending on the symptoms, age and other physical characteristics of the patient, the nature and severity of the disorder to be treated or prevented, the degree of comfort desired, the route of administration, and the concentration of the therapeutic agent in the formulation. Any of the pharmaceutical formulations described herein may be administered in a single dose or in divided doses.

An effective dose or amount, and any possible effects on the timing of administration, may need to be identified for any particular formulation (for example, based on concentration of the active agent) or method of the present invention. This may be accomplished by routine experiment as described herein. The effectiveness of any formulation and method of treatment or prevention may be assessed by administering the formulation and assessing the effect of the administration by measuring one or more indices or symptoms associated with dry eye or dry mouth to determine that the formulation reduces one or more of the indices or symptoms, and with the degree of comfort to the patient, compared to pretreatment indices or symptoms, or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of the active agents, the physiological condition of the patient (including age, sex, disease type and stage, general physical condition, responsiveness to a given dosage and type of medication), the route of administration, and the like. Guidelines may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices of ocular inflammation at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of one week from the onset of therapy, and subsequent reevaluations occurring periodically every few days or every one to two weeks during therapy and then every month thereafter. Adjustments to the amount(s) of the therapeutic agents administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

Toxicity and therapeutic efficacy may be determined by standard pharmaceutical procedures in cell cultures or experimental animals, e.g., for determining the LD₅₀ and the ED₅₀. Topical formulations have lower risk of toxicity than systemically administered formulations. However, since oxytocin has low toxicity, even when administered systemically, there is little risk of toxicity by ocular administration. Compositions that exhibit large therapeutic indices are preferred. Topical administration carries the lowest risk of toxicity compared to systemic administration.

The pharmaceutical compositions described above may additionally comprise one or more additional active ingredients defined herein to include tear substitutes, antiallergenic agents and vasoconstrictors. Such compositions may be used to treat or prevent dry eye or dry mouth or both, and they can also be used to treat an underlying or concurrent disorder or disease such as ocular allergy or other autoimmune inflammatory eye diseases or to treat or prevent symptoms accompanying dry eye/dry mouth. For example, an embodiment of a pharmaceutical composition may comprise the therapeutic neuropeptides and a tear substitute or other lubricant. Or, it may comprise a therapeutic neuropeptide and an antiallergenic agent, and optionally a tear substitute or lubricant.

Exemplary uses of various agents in treating ocular allergy and compositions thereof are described in U.S. patent application Ser. No. 10/762,201 filed Mar. 20, 2004 and U.S. patent application Ser. No. 11/074,000, filed Mar. 3, 2005, which claims priority to U.S. Provisional Patent Application 60/549,703, filed Mar. 3, 2004, all of which applications are incorporated by reference in their entireties. The dosages and combinations of agents described therein, for example, may be combined with the presently described neuropeptide formulations.

A variety of tear substitutes are known in the art and include, but are not limited to: monomeric polyols (such as glycerol, propylene glycol, and ethylene glycol); polymeric polyols (such as polyethylene glycol); cellulose esters (such as hydroxypropylmethyl cellulose, carboxy methylcellulose sodium and hydroxy propylcellulose); dextrans (such as dextran 70); water soluble proteins (such as gelatin); vinyl polymers (such as polyvinyl alcohol, polyvinylpyrrolidone, and povidone); and carbomers (such as carbomer 934P, carbomer 941, carbomer 940 and carbomer 974P). Many such tear substitutes are commercially available, which include, but are not limited to, cellulose esters such as Bion Tears®, Celluvisc®, Genteal®, OccuCoat®, Refresh®, Teargen II®, Tears Naturale®, Tears Naturale II®, Tears Naturale Free®, and TheraTears®; and polyvinyl alcohols such as Akwa Tears® HypoTears®, Moisture Eyes®, Murine Lubricating®, and Visine Tears®. Tear substitutes may also be comprised of paraffins, such as the commercially available Lacri-Lube ointments. Other commercially available ointments that are used as tear substitutes include Lubrifresh PM®, Moisture Eyes PM® and Refresh PM®.

Exemplary antihistamines include, but are not limited to, pheniramine, emedastine difumarate and levocabastine. In other embodiments, the invention features pharmaceutical compositions comprising an effective amount of a mast cell stabilizer and a tear substitute. Exemplary mast cell stabilizers include, but are not limited to, nedocromil, lodoxamide, cromolyn, and cromolyn sodium. Exemplary drugs with multiple modes of action include, but are not limited to, azelastine, epinastine, olopatadine and ketotifen fumarate.

Exemplary vasoconstrictors include, but are not limited to, naphazoline, antolazine, tetrahydozoline and oxymetazoline.

The therapeutic agents in the pharmaceutical formulations and any additional active agents such as antiallergenic agents or other active ingredients may be in the form of a pharmaceutically acceptable salt.

In some embodiments the pharmaceutical compositions will be formulated as solutions, suspensions, or ointments and other dosage forms for topical ophthalmic or oral administration in a pharmaceutically acceptable carrier, adjuvant, or vehicle. Aqueous solutions are generally preferred, based on ease of formulation, as well as a patient's ability to easily administer such compositions by means of instilling one to two drops of the solutions in the affected eyes or used as an eye wash. For dry mouth formulations may be sprayed in the mouth or used as a mouth wash. However, the compositions may also be suspensions, viscous or semi-viscous gels, or other types of solid or semi-solid compositions.

Any of a variety of carriers may be used in the topical formulations of the present invention, including water, mixtures of water and water-miscible solvents (such as C₁- to C₇-alkanols), vegetable oils or mineral oils comprising from 0.5 to 5% non-toxic water-soluble polymers, natural products (such as gelatin, alginates, pectins, tragacanth, karaya gum, xanthan gum, carrageenin, agar and acacia), starch derivatives (such as starch acetate and hydroxypropyl starch), and also other synthetic products (such as polyvinyl alcohol, polyvinylpyrrolidone, polyvinyl methyl ether, or polyethylene oxide—preferably cross-linked polyacrylic acid, such as neutral Carbopol), or mixtures of those polymers. The concentration of the carrier is, typically, from 1 to 100,000 times the concentration of the active ingredient.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ or portion of the body, to another organ or portion of the body. Each carrier must be “acceptable” in the sense of being compatible with the other ingredients of the supplement and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

Additional non-active ingredients that may be included in the formulations include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0 or 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred. Generally, buffers will be used in amounts ranging from about 0.05 to 2.5 percent by weight, and preferably, from 0.1 to 1.5 percent.

Tonicity is adjusted if needed typically by tonicity enhancing agents. Such agents may, for example, be of ionic or non-ionic type. Examples of ionic tonicity enhancers are alkali metal or earth metal halides, such as, for example, CaCl₂, KBr, KCl, LiC1, NaI, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

In certain embodiments, the topical formulations additionally comprise a preservative. A preservative may typically be selected from a quaternary ammonium compound such as benzalkonium chloride, benzoxonium chloride or the like. Benzalkonium chloride is better described as N-benzyl-N—(C₈-C₁₈ alkyl)-N,N-dimethylammonium chloride. Examples of preservatives different from quaternary ammonium salts are alkyl-mercury salts of thiosalicylic acid, such as, for example, thiomersal (thimerosol), phenylmercuric nitrate, phenylmercuric acetate or phenylmercuric borate; sodium perborate; sodium chlorite; parabens, such as, for example, methylparaben or propylparaben; alcohols, such as, for example, chlorobutanol, benzyl alcohol or phenyl ethanol; guanidine derivatives, such as, for example, chlorohexidine or polyhexamethylene biguanide; sodium perborate; or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the topical formulations of this invention do not include a preservative. Such formulations would be useful for patients who wear contact lenses, or those who use several topical ophthalmic drops and/or those with an already compromised ocular surface wherein limiting exposure to a preservative may be more desirable.

The topical formulation may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements.

Other compounds may also be added to the formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family, vinyl polymers, and acrylic acid polymers.

The topical formulations of the present invention may be packaged as either a single dose product or a multi-dose product. The “dose” for an ophthalmic formulation of the invention is typically 2-3 drops per eye and can be calculated based on the concentration of active agent in the formulation and number of drops as described above. Doses of oral formulations are similarly calculated. The single dose product is sterile prior to opening of the package and all of the composition in the package is intended to be consumed in a single application to one or both eyes of a patient. The use of an antimicrobial preservative to maintain the sterility of the composition after the package is opened is generally unnecessary.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an antimicrobial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye/dry mouth conditions or pre-existing ocular or oral irritation. However, the single dose packaging arrangements currently available, such as small volume plastic vials prepared by means of a process known as “form, fill and seal,” have several disadvantages for manufacturers and consumers.

While the formulations of this invention can be formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

### 7. ALTERNATIVES AND EXTENSIONS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### 8. REFERENCES

1. Moss S E, Klein R, Klein B E. Incidence of dry eye in an older population. Archives of ophthalmology 2004; 122(3):369-73.
2. Marsh P, Pflugfelder S C. Topical nonpreserved methylprednisolone therapy for keratoconjunctivitis sicca in Sjogren syndrome. Ophthalmology 1999; 106(4):811-6.
3. Stevenson D, Tauber J, Reis B L. Efficacy and safety of cyclosporin A ophthalmic emulsion in the treatment of moderate-to-severe dry eye disease: a dose-ranging, randomized trial. The Cyclosporin A Phase 2 Study Group. Ophthalmology 2000; 107(5):967-74.
4. Lemp M A, Nichols K K. Blepharitis in the United States 2009: a survey-based perspective on prevalence and treatment. The ocular surface 2009; 7(2 Suppl):S1-S14.
5. Nemet A Y, Vinker S, Kaiserman I. Associated morbidity of blepharitis. Ophthalmology 2011; 118(6):1062-8.
6. Perrier C, Rutgeerts P. Cytokine blockade in inflammatory bowel diseases. Immunotherapy 2011; 3(11):1341-52.
7. Welch M G, Anwar M, Chang C Y, et al. Combined administration of secretin and oxytocin inhibits chronic colitis and associated activation of forebrain neurons. Neurogastroenterology and motility: The official journal of the European Gastrointestinal Motility Society 2010; 22(6):654-e202.
8. Yoon K C, Jeong I Y, Park Y G, Yang S Y. Interleukin-6 and tumor necrosis factor-alpha levels in tears of patients with dry eye syndrome. Cornea 2007; 26(4):431-7.
9. Narayanan S, Corrales R M, Farley W, et al. Interleukin-1 receptor-1-deficient mice show attenuated production of ocular surface inflammatory cytokines in experimental dry eye. Cornea 2008; 27(7):811-7.
10. De Paiva C S, Volpe E A, Gandhi N B, et al. Disruption of TGF-beta signaling improves ocular surface epithelial disease in experimental autoimmune keratoconjunctivitis sicca. PLoS One 2011; 6(12):e29017.
11. Viau S, Maire M A, Pasquis B, et al. Time course of ocular surface and lacrimal gland changes in a new scopolamine-induced dry eye model. Graefes Arch Clin Exp Ophthalmol. 2008 June; 246(6): 857-67.
12. Araki-Sasaki, K et al. An SV40-Immortalized Human Corneal Epithelial Cell Line and Its Characterization. IOVS 1995; 36: 614-621.
13. Diebold, Y et al. Characterization of a Spontaneously Immortalized Cell Line (IOBA-NHC) from Normal Human Conjunctiva. IOVS 2003; 44: 4263-74

1. A pharmaceutical composition formulated for topical oral or ophthalmic use comprising a therapeutically or prophylactically effective amount of secretin, or a combination of oxytocin and secretin, or a combination of oxytocin and vasopressin and secretin, or a combination of oxytocin and vasopressin, or a combination of secretin and vasopressin, or biologically active fragments, analogs or derivatives thereof, for the treatment of dry mouth or dry eye.

2. The pharmaceutical composition of claim 1, wherein the therapeutically or prophylactically effective amount of secretin, oxytocin, or vasopressin or biologically active fragment, analog or variant thereof is in the range of from about 0.0001 to 0.005 mg/ml, 0.005 to 0.05 mg/ml, 0.05 to 0.5 mg/ml, 0.5 to 1 mg/ml, and 1 to 5 mg/ml.

3. The pharmaceutical composition of claim 1, wherein the pharmaceutical composition further comprises a tear substitute or other lubricant.

4. The pharmaceutical composition of claim 1, further comprising at least one antiallergenic agent.

5. The pharmaceutical composition of claim 1, further comprising at least one antiallergenic agent and a tear substitute or other lubricant.

6. A method of treating or preventing dry eye in a subject, comprising administering to the eye surface of the subject a therapeutically or prophylactically effective amount of secretin, or a combination of oxytocin and secretin, or a combination of oxytocin and vasopressin and secretin, or a combination of oxytocin and vasopressin, or a combination of secretin and vasopressin or biologically active fragments, analogs or derivatives thereof.

7. A method of treating or preventing dry mouth in a subject, comprising administering to the oral cavity of the subject a therapeutically or prophylactically effective amount of secretin, or oxytocin, or vasopressin, or a combination thereof or biologically active fragments, analogs or derivatives thereof.

8. The method of claim 6, wherein the dry eye is associated with a disease or disorder selected from the group comprising dry eye syndrome, blepharitis, meibomitis, ocular rosacea, thyroid eye disease, chronic papillary conjunctivitis, chronic follicular conjunctivitis, nonspecific chronic conjunctivitis, giant papillary conjunctivitis, ocular cicatricial pemphigoid, cicatrizing conjunctivitis, allergic conjunctivitis, phlyctenular corneoconjunctivitis, episcleritis, diffuse scleritis, nodular scleritis, scleromalacia perforans, superficial punctate keratitis, infectious keratitis, peripheral ulcerative keratitis, Thygeson's superficial punctate keratitis, corneal graft rejection, disciform keratitis, stromal keratitis, anterior uveitis, HLA-B27 uveitis, Parkinson's disease, Behcet's disease, atopic eye disease, pars planitis, sarcoidosis, sympathetic ophthalmia, Fuch's heterochromic iridocyclitis, glaucomatocyclitis crisis (Posner-Schlossman syndrome), chronic macular edema, cystoid macula edema, central serous choreoretinopathy, white dot syndrome, anemia, hypertension, stroke, mumps, acute retinal necrosis, and autoimmune diseases comprising juvenile rheumatoid arthritis, lupus erythematosus, cystic fibrosis, Sjogren's syndrome, Vogt-Koyanagi-Harada syndrome, HIV/AIDS, and diabetes.

9. The method of claim 7, wherein the dry mouth is associated with a disease or disorder selected from the group comprising inflammatory disorders, HIV/AIDS, Alzheimer's disease, diabetes, anemia, hypertension, Parkinson's disease, stroke, mumps, and autoimmune diseases comprising juvenile rheumatoid arthritis, cystic fibrosis, rheumatoid arthritis, lupus erythematosus and Sjogren's syndrome.

10. The method of claim 6, wherein the secretin, or combination of oxytocin and secretin, or a combination of oxytocin and vasopressin and secretin, or a combination of oxytocin and vasopressin, or a combination of secretin and vasopressin or biologically active fragments, analogs or derivatives thereof is administered multiple times per day.

11. The method of claim 7, wherein the secretin, or oxytocin, or vasopressin, or combination thereof or biologically active fragments, analogs or derivatives thereof is administered multiple times per day.

12. The method of claim 6, wherein the combination of oxytocin and secretin, or a combination of oxytocin and vasopressin and secretin, or a combination of oxytocin and vasopressin, or a combination of secretin and vasopressin or biologically active fragments, analogs or derivatives thereof is administered as a single pharmaceutical composition.

13. The method of claim 7 wherein the combination is administered as a single pharmaceutical composition.

14. The method of claim 6, wherein the therapeutically effective amount is an amount from about 0.0001-0.005 mg/ml, 0.005 to 0.05 mg/ml; 0.05 to 0.5 mg/ml, 0.5 to 1 mg/ml, and 1 to 5 mg/ml.

15. The pharmaceutical composition of claim 1, wherein the composition is an aqueous solution or an ointment.

16. The method of claim 7, wherein the therapeutically effective amount is an amount from about 0.0001-0.005 mg/ml, 0.005 to 0.05 mg/ml; 0.05 to 0.5 mg/ml, 0.5 to 1 mg/ml, and 1 to 5 mg/ml.
